(19) United States
(12) Patent Application Publication
(10) Pub. No.: US 2015/0157651 A1
(43) Pub. Date: Jun. 11, 2015

(54) TRI-SUBSTITUTED GLYCEROL COMPOUNDS FOR USE IN THE TREATMENT OF CLINICALLY ISOLATED SYNDROME AND/OR MULTIPLE SCLEROSIS

(71) Applicants: ALPHAPTOSE GMBH, Hamburg (DE); UNIVERSITÄTSKLINIKUM HAMBURG-EPPENDORF, Hamburg (DE)

(72) Inventors: Pierre Abramowski, Hamburg (DE); Roland Martin, Zurich (CH)

(21) Appl. No.: 14/401,934

(22) PCT Filed: May 21, 2013
(86) PCT No.: PCT/EP2013/060431

§ 371 Date: Nov. 18, 2014

(30) Foreign Application Priority Data
May 18, 2012 (GB) 1208850.6

(51) Int. Cl. A61K 31/685; A61K 45/06

(52) U.S. Cl. CPC A61K 31/685; A61K 45/06

(57) ABSTRACT

The present invention relates to tri-substituted glycerol compounds according to formula (I) as described herein for use in the treatment of clinically isolated syndrome, relapsing remitting multiple sclerosis (RR-MS) and/or secondary progressive multiple sclerosis (SP-MS). The tri-substituted glycerol compounds according to formula (I) as described herein may also be used for the treatment of multiple sclerosis patients not adequately responding to interferon therapy. The present invention also relates to a tri-substituted glycerol compound as described herein, for use as a medicament, wherein the tri-substituted glycerol compound is administered in combination with at least one further pharmaceutically active compound.

The present invention relates to tri-substituted glycerol compounds according to formula (I) as described herein for use in the treatment of clinically isolated syndrome, relapsing remitting multiple sclerosis (RR-MS) and/or secondary progressive multiple sclerosis (SP-MS). The tri-substituted glycerol compounds according to formula (I) as described herein may also be used for the treatment of multiple sclerosis patients not adequately responding to interferon therapy. The present invention also relates to a tri-substituted glycerol compound as described herein, for use as a medicament, wherein the tri-substituted glycerol compound is administered in combination with at least one further pharmaceutically active compound.

### INTRODUCTION

Tri-substituted glycerol compounds belong to the class of synthetic ether-linked alkyl-lysophospholipids, which are known to have an anti-cancerogenic activity. For this reason they are also collectively named “anti-tumor ether lipids” (reviewed, e.g., by Arthur, G., and Bittman, R. (1998) Biochim. Biophys. Acta 1390, 85-102; Jendrossek, V., and Handrick, R. (2003) Curr. Med. Chem. Anti-Canc. Agents 3, 343-353; Mollinedo, F. et al. (2004) Curr. Med. Chem. 11, 3163-3184). Unlike most conventional chemotherapeutic drugs, synthetic ether lipids do not directly target cellular DNA but rather affect the plasma membrane lipid composition and/or interfere with various signal transduction pathways.

Racemic 1-O-octadecyl-2-O-methyl-sn-glycero-3-phosphocholine (also referred to as ET-18-OCH3, AP-121 or edelfosine) is considered to be the prototype of the anti-tumor ether lipids. 1-O-octadecyl-2-O-methyl-sn-glycero-3-phosphocholine represents a synthetic analogue of the platelet activating factor (PAF; 1-O-alkyl-2-acetyl-sn-glycero-3-phosphocholine), a potent phospholipid activator and mediator of many leukocyte functions, including platelet aggregation, inflammation, and anaphylaxis.

Aside from their anti-tumor activity, the above ether lipids are believed to be involved in a variety of other physiological processes such as inflammation, the immune response or allergic reactions. Some ether lipids have been suggested as candidate compounds for the treatment of various immune diseases (cf., for example, the International Patent Applications WO 87/01257 and WO 90/14829, respectively).

Multiple sclerosis (MS) is an autoimmune disease of the central nervous system (CNS) which is characterized e.g. by inflammatory lesions in the CNS, demyelation and scarring, axonal loss and atrophy. Animal and human studies point to a central role of autoreactive CD4+ cells in MS pathogenesis. MS may thus be considered to be a CD4+ T cell-mediated autoimmune disease.

Different subtypes of MS characterized by different disease courses have been described, e.g. relapsing-remitting MS (RR-MS), primary-progressive MS (PP-MS), secondary-progressive MS (SP-MS) and progressive-relapsing MS (PR-MS). While currently some treatments for MS such as interferon therapy exist, most of them treatments have drawbacks, such as unwanted side effects or inconvenient modes of administration. Hence, there remains a need for compounds useful in the treatment of MS.

### SUMMARY OF THE INVENTION

In a first aspect the present invention relates to a tri-substituted glycerol compound according to formula (I)

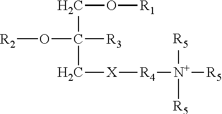

or an enantiomer or diastereomer or a pharmaceutically acceptable salt thereof for use in the treatment of clinically isolated syndrome (CIS), relapsing remitting multiple sclerosis (RR-MS) and/or secondary progressive multiple sclerosis (SP-MS), wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl.

In another aspect, the present invention relates to a tri-substituted glycerol compound according to formula (I)

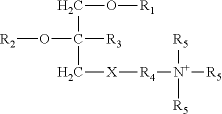

or an enantiomer or diastereomer or a pharmaceutically acceptable salt thereof for use in the treatment of multiple sclerosis patients not adequately responding to interferon therapy, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl.

In one embodiment, the present invention relates to a tri-substituted glycerol compound for use in the treatment of clinically isolated syndrome, relapsing remitting multiple sclerosis (RR-MS) and/or secondary progressive multiple sclerosis (SP-MS), wherein X is phosphate, R₁ is —(CH₂)₁₇—CH₃, R₂ is CH₃, R₃ is H, R₄ is —(CH₂)₂—, and R₅ is CH₃.

In a further embodiment, the present invention relates to a tri-substituted glycerol compound for use in the treatment of multiple sclerosis patients not adequately responding to interferon therapy, wherein X is phosphate, R₁ is —(CH₂)₁₇—CH₃, R₂ is CH₃, R₃ is H, R₄ is —(CH₂)₂—, and R₅ is CH₃.

In another embodiment, the present invention relates to the tri-substituted glycerol compound for any of the above described uses, wherein the tri-substituted glycerol compound is administered in an amount of 10-40 mg/day, optionally 20-30 mg/day

A further embodiment of the invention relates to the tri-substituted glycerol compound for any of the above described uses, wherein the tri-substituted glycerol compound is administered in combination with at least one further pharmaceutically active compound.

Another embodiment of the invention relates to the tri-substituted glycerol compound for any of the above described uses, wherein the at least one further pharmaceutically active compound is selected from the group consisting of
glucocorticoids, such as cortisone, prednisone, prednisolone and methyl-prednisolone;
interferone-beta compounds, such as interferone-beta 1a and interferone-beta 1b;
immunosuppressive agents, such as mitoxantrone, cyclophosphamid, methotrexat, azathioprine, mycophenolate, cladribine and fingolimod;
immunomodulatory compounds such as glatiramer acetate (GA), dirucotide, laquinimod and teriflunomid;
monoclonal antibodies, such as natalizumab, daclizumab, alemtuzumab and rituximab;
intravenous immunoglobulin (IVIG); and
oral fumarate.

In a further embodiment the invention relates to the tri-substituted glycerol compound for any of the above described uses, wherein the at least one further pharmaceutically active compound is selected from teriflunimod, laquinimod or oral fumarat.

Another embodiment of the invention is directed to the tri-substituted glycerol compound for any of the above uses, wherein the tri-substituted glycerol compound is administered orally.

In another aspect the present invention relates to a tri-substituted compound according to formula (I)

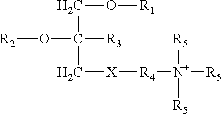

or an enantiomer or diastereomer or a pharmaceutically acceptable salt thereof for use as a medicament, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl, and
wherein the tri-substituted glycerol compound is administered in combination with at least one further pharmaceutically active compound selected from the group consisting of
glucocorticoids, such as cortisone, prednisone, prednisolone and methyl-prednisolone;
interferone-beta compounds, such as interferone-beta 1a and interferone-beta 1b;
immunosuppressive agents, such as mitoxantrone, cyclophosphamid, methotrexat, azathioprine, mycophenolate, cladribine and fingolimod;
immunomodulatory compounds such as glatiramer acetate (GA), dirucotide, laquinimod and teriflunomid;
monoclonal antibodies, such as natalizumab, daclizumab, alemtuzumab and rituximab;
intravenous immunoglobulin (IVIG); and
oral fumarate.

One embodiment of the invention relates to the tri-substituted compound according to formula (I) as defined above wherein X is phosphate, R₁ is —(CH₂)₁₇—CH₃, R₂ is CH₃, R₃ is H, R₄ is —(CH₂)₂—, and R₅ is CH₃ for use as a medicament, wherein the tri-substituted glycerol compound is administered in combination with at least one further pharmaceutically active compound as defined above.

A further embodiment of the invention relates to the the tri-substituted compound according to formula (I) as defined above for use as a medicament, wherein the tri-substituted compound is administered orally and, wherein the tri-substituted glycerol compound is administered in combination with at least one further pharmaceutically active compound as defined above.

Another embodiment of the invention relates to the tri-substituted glycerol compound according to formula (I) as defined above for use as a medicament, wherein the tri-substituted glycerol compound is administered as a solid dosage form, optionally selected from tablets, pills, capsules, and granules and, wherein the tri-substituted glycerol compound is administered in combination with at least one further pharmaceutically active compound as defined above.

One embodiment of the invention relates to the tri-substituted glycerol compound according to formula (I) as defined above for use as a medicament, wherein the tri-substituted glycerol compound is present in an amount of 10-40 mg/day, optionally 20-30 mg/day and wherein the tri-substituted glycerol compound is administered in combination with at least one further pharmaceutically active compound as defined above.

A further embodiment relates to the tri-substituted glycerol according to formula (I) compound as defined above for use as a medicament, wherein the medicament is for the treatment of multiple sclerosis and wherein the tri-substituted glycerol compound is administered in combination with at least one further pharmaceutically active compound as defined above.

Another embodiment of the invention relates to the tri-substituted glycerol compound according to formula (I) as defined above for use as a medicament, wherein the medicament is for the treatment of clinically isolated syndrome, relapsing remitting multiple sclerosis (RR-MS), secondary progressive multiple sclerosis (SP-MS) and/or multiple sclerosis patients not adequately responding to interferon therapy and wherein the tri-substituted glycerol compound is administered in combination with at least one further pharmaceutically active compound as defined above.

### FIGURE DESCRIPTION

FIG. 1: Human T-cell proliferation was affected by edelfosine. (A) Reduced PBMC proliferation upon addition of edelfosine on cell seeding was independent of the addition of PHA (phytohemagglutinin) Notably, PHA-activated cells appeared to be susceptible to edelfosine at 10-fold lower concentrations. (B) The inhibitory effect of edelfosine was also observed if the drug was added to already activated, proliferating T cells. Here, a significant reduction of proliferation in unstimulated cells was only detectable with 33.3 μg/ml edelfosine. (C) Preincubation of PBMCs with at least 3.3 μg/ml edelfosine interfered with the cells' capacity to proliferate upon PHA stimulation. No effect was detected in preconditioned, but unstimulated cells (experiments A-C: sample size n=3 donors, each approach was seeded in triplicates). (D) 1 μg/ml edelfosine or higher concentrations profoundly diminished proliferation in MBP₍₈₃₋₉₉₎-specific TCLs (T-cell line). One representative TCL of two is shown. Cells were incubated in quadruplicates. (E) PBMCs were cultured without addition of stimulus. Proliferation was detectable after seven days. The presence of anti-HLA-DR- and anti-MHC class I-blocking antibodies or 3.3 μg/ml edelfosine inhibited cellular proliferation. Bars represent mean values±SEM, *P<0.05, **P<0.01 and ***P<0.001 after post-hoc analysis.

FIG. 2: Modulated gene expression in CD4+ T cells upon culture with edelfosine. A signal log ratio (SLR)≧0.8 for upregulated genes or ≦−0.8 for downmodulated genes was set as a cut-off to determine genes whose expressions were significantly modulated after t-test analysis.

FIG. 3: Gene expression analysis allowed clustering of up- or downregulated genes to determine biological pathways affected by edelfosine in human CD4+ T cells. (A) The incubation of unstimulated cells with 10 μg/ml edelfosine for 30 h resulted in the upregulation of apoptosis- and cell death-associated genes. Genes involved in immune response and antigen processing and presentation were downregulated. (B) In the case of stimulated cells which were cultured in presence of 3.3 μg/ml edelfosine the downmodulation of cell-cycle progression-related genes was found. Additionally, the incubation with edelfosine resulted in the upregulation of genes assigned to immune response- and virus response-pathways characterized by type I interferon-regulated genes.

FIG. 4: Gene list summary of antigen processing and presentation (MHC class II)-as well as immune response (type I interferon)-associated genes. (A) Unstimulated human CD4+ T cells were cultured with 10 μg/ml edelfosine. 25 downregulated genes were assigned to biological pathways for antigen processing and presentation (SLR≦−0.8). (B) Stimulated, 3.3 μg/ml edelfosine-treated CD4+ T cells demonstrated upregulation of genes involved in biological processes of immune response and response to virus (SLR≧0.6).

FIG. 5: Modulation of gene expression in human CD4+ T cells mediated by stimulation and edelfosine addition. (A) The incubation of cells in absence of a stimulus resulted in an edelfosine concentration-dependent downregulation of antigen processing- and presentation-associated genes. (B) The activation of cells with beads coated with antibodies against CD2, CD3 and CD28 in presence of 3.3 μg/ml edelfosine resulted in a consistent upregulation of immune and virus response-associated genes. The values of differential gene-expression changes correspond to the SLR (red for upregulation, blue for downregulation). Genes are clustered hierarchically in the dendrogram over the expression matrix. The height of the branches is inversely proportional to the degree of neighborhood between clusters (images generated with R statistical platform 2.12, gplots package 2.8.0).

FIG. 6 describes the influence of edelfosine on EAE disease course. Our aim was to study the effect of edelfosine on the immune system in the context of experimental autoimmune encephalomyelitis (EAE), an animal model of multiple sclerosis. In the course of EAE CD4+ T cell and mononuclear cell inflammation is followed by axonal damage and demyelination in the central nervous system (CNS) which results in progressive hind-limb paralysis. In order to analyze the anti-inflammatory properties of edelfosine we have used the relapsing-remitting EAE (RR-EAE) model in SJL mice. We also examined the edelfosine impact on human T-cell proliferation. For EAE induction SJL mice were immunized with the proteolipid PLP₍₁₃₉₋₁₅₁₎. Edelfosine was adminstered by intraperitoneal injection from day 0 at a daily basis. EAE disease course was assessed by clinical scoring in a 0-5 scale until day 40 (*P<0.05, 2-way ANOVA, n=7). To investigate the therapeutic effect of edelfosine on RR-EAE disease course, SJL mice were immunized and treatment of the mice was started at disease onset by intraperitoneal injection from day 11 at a daily basis until day 45.

FIG. 7 describes the impact of edelfosine on proliferation of antigen-specific cells. To examine the influence of edelfosine on antigen-specific cells in the periphery lymph nodes from SJL mice were prepared for thymidine proliferation assays 9 days after immunization. Cells were restimulated ex vivo by addition of mitogenic- (a), polyclonal or disease-relevant (PLP₍₁₃₉₋₁₅₁₎; b) antigen. Lymph node cells of edelfosine-treated mice retained their proliferative function. Re-activation of cells with PLP₍₁₃₉₋₁₅₉₎ resulted in an edelfosine dose-dependent proliferation. Notably, antigen-induced proliferation appeared to be accompanied by a treatment-dependent background proliferation resulting in increased SI upon edelfosine treatment compared to PBS treatment of mice (c, d).

FIG. 8 describes the effect of edelfosine on CNS cellular infiltration in EAE acute phase. SJL mice were immunized with PBS₍₁₃₉₋₁₅₁₎. 15 days after EAE induction CNS cells were analyzed by flow cytometry. The administration of 10 mg/kg edelfosine reduced the absolute number of CNS-infiltrating CD45+ leukocytes (a). We observed a relative decrease in infiltrating CD4+ T cells if mice were treated with 10 mg/kg edelfosine whereas frequencies of CD8+ T cells appeared to be not affected (b). Edelfosine treatment induces a dose-dependent activation of caspase 3 in CD4+ T cells (c). We found a significant treatment effect with a significant difference between 10 mg/kg edelfosine and PBS treatment in CD4+ cells (*P<0.05 compared to PBS control group, post-hoc comparison after 1-way ANOVA, n=4). Similar differences were found for T cells in spleens at day 9 after immunization.

FIG. 9 describes the influence of edelfosine on human T cell proliferation. Analysis of non-stimulated PBMCs after 24 h by flow cytometry pointed to the induction of non-apoptotic cell death if 10 μg/ml of 33.3 μg/ml edelfosine were applied (a). PBMCs were used for the examination of concentration-dependent edelfosine impact on human T cell activation and proliferation (b, 72 h of co-culture). A reduction in proliferation was already detectable upon addition of 1.0 μg/ml edelfosine (**P<0.01, n=3). Enriched CD4+ T cells from PBMCs of both two age-matched female and male donors were incubated for 30 h in presence of 3.3 μg/ml edelfosine. Cells were stimulated with beads coated with antibodies against CD2, CD3 and CD28. Gene expression analysis revealed an edelfosine-mediated upregulation of genes associated with apoptosis and cell death. On the contrary, cell cycle-associated genes were found to be down-regulated.

### CONCLUSIONS

Prophylactic as well as therapeutic daily treatment of SJL mice with 10 mg/kg edelfosine led to EAE amelioration.
Edelfosine interfered with PLP₍₁₃₉₋₁₅₁₎ specific immune cells in vivo thereby affecting their proliferative capacity in restimulation experiments ex vivo.
Treatment of EAE affected mice with 10 mg/kg edelfosine increased the activation of caspase 3 in CNS infiltrating CD4+ T cells.
Our results imply that edelfosine may ameliorate EAE by acting on disease-specific immune cells, possibly via apoptosis induction.
Gene expression analysis of stimulated, edelfosine-treated human CD4+ T cells supported the proposed cytostatic, apoptosis-inducing action of edelfosine.
Edelfosine may affect the antigen-specific activation of T cells and it will be interesting to study its impact on immunological synapse formation.

### DETAILED DESCRIPTION OF THE INVENTION

Currently existing MS treatments mainly target inflammation and also include the use of immunosuppressants and/or immunomodulators. Glucocorticoids are used in acute phases of the disease. Glatiramer acetate (GA), Interferone-beta compounds or fingolimod are usually used as first-line treatments of MS, while mitoxantrone and natalizumab may be applied in escalation therapies. Interferone-beta formulations are frequently used in the treatment of RR-MS. However, interferone-beta therapy also includes some drawbacks such as the modest impact on MS progression, the frequency of subcutaneous injections, side effects such as flu-like symptoms, expensive recombinant production and the existence of interferone-beta non-responders even after initial responsiveness (antibody producers). Glatimer acetate which is also used in first-line treatment is sold under the trade name Copaxone. Copaxone is a copolymer comprising alanine, lysine, glutamic acid and tyrosine and is administered by subcutaneous injection. However, a number of MS patients refuse injectable treatments per se or due to needle phobia. Treatment with mitoxantrone is also associated with side-effects such as the risk of inducing therapy-related leukemia and cardiotoxicity, while treatment with natalizumab may lead to progressive multifocal leukoencephalopathy.

As currently existing therapies include drawbacks, there remains a need for further treatment options for patients suffering from clinically isolated syndrome (CIS), relapsing remitting multiple sclerosis (RR-MS) and/or secondary progressive multiple sclerosis (SP-MS). Furthermore, while interferon-beta is most frequently used for the treatment of RR-MS patients, there are still patients who do not respond to this therapy (i.e. so-called non-responders) or turning into non-responders in the course of therapy. Hence, there also remains a need for a replacement therapy for patients not adequately responding to interferone-beta therapy.

It has now been found that tri-substituted glycerol compounds according to formula (I) as described herein may be used in the treatment of early stages of MS, e.g. in the treatment of patients suffering from clinically isolated syndrome (CIS), relapsing remitting multiple sclerosis (RR-MS) and/or secondary progressive multiple sclerosis (SP-MS). The tri-substituted glycerol compounds according to formula (I) may also be administered orally and/or at low doses thus allowing easier and more convenient administration compared to injection. Furthermore, the tri-substituted glycerol compounds disclosed may also be used in the treatment of non-responders to interferon-beta therapy.

In a first aspect the present invention thus relates to a tri-substituted glycerol compound according to formula (I)

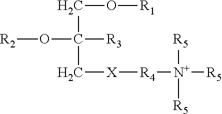

or an enantiomer or diastereomer or a pharmaceutically acceptable salt thereof for use in the treatment of clinically isolated syndrome (CIS), relapsing remitting multiple sclerosis (RR-MS) and/or secondary progressive multiple sclerosis (SP-MS), wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl.

One specific aspect of the invention relates to the tri-substituted glycerol compound according to formula (I) as defined herein or an enantiomer or diastereomer or a pharmaceutically acceptable salt thereof for use in the treatment of clinically isolated syndrome.

Another specific aspect of the invention relates to the tri-substituted glycerol compound according to formula (I) as defined herein or an enantiomer or diastereomer or a pharmaceutically acceptable salt thereof for use in the treatment of relapsing remitting multiple sclerosis (RR-MS).

A further specific aspect of the invention relates to the tri-substituted glycerol compound according to formula (I) as defined herein or an enantiomer or diastereomer or a pharmaceutically acceptable salt thereof for use in the treatment of secondary progressive multiple sclerosis (SP-MS).

As used herein “clinically isolated syndrome” refers to patients suffering from a first clinical sign or syndrome which could be consistent with MS. Such a first clinical sign is a neurological episode lasting at least 24 hours and caused by inflammation and/or demyelination in one or more sites in the central nervous system (see e.g. National Multiple Sclerosis Society; http://www.nationalmssociety.org/about-multiple-sclerosis/what-we-know-about-ms/diagnosing-ms/cis/index.aspx). Patients diagnosed with clinically isolated syndrome may but do not have to develop multiple sclerosis. CIS may thus represent an early stage of multiple sclerosis.

With respect to multiple sclerosis four different disease courses have been described, namely, relapsing remitting MS (RR-MS), primary progressive MS (PP-MS), secondary progressive MS (SP-MS) and progressive-relapsing MS (PR-MS). RR-MS is characterized by episodes of acute worsening of the neurologic function followed by periods with variable degree of recovery. However, the disease course between the episodes is stable. This initial RR-MS may be followed by a progressively worsening disease course, i.e. the so-called SP-MS. In contrast to RR-MS, PR-MS also shows relapses, however the periods between these relapses are characterized by continuing progression of the disease. Finally, PP-MS shows a disease course with gradual, almost continuous worsening with no distinct relapses (Lublin F. D.; MS in focus, Issue 14-2009, Introduction to the disease courses of MS; http://www.msif.org/en/resources/msif_resources/msif_publications/ms_in_focus/iss ue_{—}14_disease_courses_in_ms/intro_disease_co.html).

Homeostatic T-cell proliferation has been shown to be upregulated in MS patients. It has now been found that the tri-substituted glycerol compounds according to formula (I) as described herein e.g. in non-toxic and non-lytic doses, are not only capable to inhibit T-cell proliferation after mitogenic and antigen-specific activation but may also inhibit the homeostatic T-cell proliferation (i.e. the “background” proliferation under conditions without stimulation). Thus, the tri-substituted glycerol compounds according to formula (I) as described herein may be used for treatment in an early stage of the disease and/or at low doses.

Low doses as used herein may be doses which are lower than the doses used in tumor therapy. Exemplary low doses are 10-40 mg/day, optionally 20-30 mg/day of the tri-substituted glycerol compound as described herein. Further exemplary low doses are 10-15 mg/day or 10-20 mg/day or 10-25 mg/day or 15-25 mg/day.

Treatment of multiple sclerosis in early stages of the disease may inter alia avoid damages occurring in the CNS, e.g. lesions. Hence, in some embodiments of the invention, the tri-substituted glycerol compound as described herein is used for the treatment of patients in an early stage of their disease, i.e. patients having clinically isolated syndrome, an early stage of relapsing remitting multiple sclerosis (RR-MS) and/or an early stage of secondary progressive multiple sclerosis (SP-MS). In another embodiment of the invention, the tri-substituted glycerol compound as described herein is used for the treatment of a patient not adequately responding to interferon therapy and having clinically isolated syndrome, an early stage of RR-MS and/or an early stage of SP-MS.

As described above, patients having clinically isolated syndrome have not already been diagnosed for multiple sclerosis but are at risk of developing multiple sclerosis and CIS may thus represent an early stage of MS. Patients in an early stage of RR-MS within the meaning of the invention includes patients having only recently been diagnosed for RR-MS due to the occurrence of a second relapse. Patients in an early stage of SP-MS within the meaning of the invention includes patients wherein the RR-MS disease course has only recently turned into an SP-MS course. Furthermore, the term “patients in an early stage of their disease” within the meaning of the present invention includes patients having an EDSS under 4. EDSS (Expanded Disability Status Scale) is used to quantify the disability in multiple sclerosis. The EDSS ranges from 0 to 10, wherein 0 stands for a normal neurological exam while 10 indicates death due to multiple sclerosis. Furthermore, patients in an early stage of their disease also includes patients in the first 10 years after MS diagnosis.

It has further been found that type I interferon-associated genes are upregulated if cells are stimulated with a tri-substituted glycerol compound according to the invention such as edelfosine. These genes were also shown to be upregulated in a study of gene-expression profiles of peripheral blood mononuclear cells (PBMC) derived from MS patients treated with interferon-beta (Serrano-Fernández et al. (2010), Time course transcriptomics of IFNB1b drug therapy in multiple sclerosis, Autoimmunity 43:172-8). Hence, the tri-substituted glycerol compounds according to the invention may be used as replacement therapy for patients not adequately responding to interferone beta therapy. Patients not adequately responding to interferone beta therapy may also be referred to as non-responders.

In another aspect, the present invention relates to a tri-substituted glycerol compound according to formula (I)

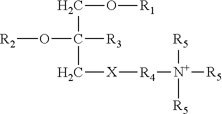

or an enantiomer or diastereomer or a pharmaceutically acceptable salt thereof for use in the treatment of multiple sclerosis patients not adequately responding to interferon therapy, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl.

Patients not adequately responding to interferon therapy include patients not responding to interferon-beta therapy, in particular patients not responding to interferon-beta 1a or interferone-beta 1b therapy. Furthermore, the patient may have been initially been a non-responder to interferon therapy or developed to a non-responder to the interferon therapy in the course of treatment. In one embodiment of the invention the patient not responding to interferon therapy is a patient diagnosed with CIS. In another embodiment of the invention, the patient not adequately responding to interferon therapy is a patient diagnosed with RR-MS. In a further embodiment of the invention, the patient not adequately responding to interferon therapy is a patient diagnosed with SP-MS. Patients not adequately responding to interferon therapy include patients having developed neutralizing antibodies against interferon treatment.

The tri-substituted glycerol compound as used herein may be present in amorphous or in crystalline form. The term “amorphous”, as used herein, refers to a solid in which there is no long-range order of the positions of the atoms, i.e. a non-crystalline material. In some embodiments of the invention, the tri-substituted glycerol compound is present in crystalline form.

The terms “Cₙ alkyl”, “Cₙ hydroxyalkyl”, and “Cₙ cycloalkyl”, as used herein, denote an alkyl group, a hydroxyalkyl group or a cycloalkyl group having n carbon atoms, respectively. For example, the term “C₁₈ alkyl” refers to an alkyl group having 18 carbon atoms. If ranges (such as C₁-C₃ alkyl) are mentioned, these ranges also disclose explicitly each single compound falling within the range (i.e. C₁, C₂, C₃). The alkyl groups or hydroxyalkyl groups according to the invention may be straight or branched.

The tri-substituted glycerol compounds of formula (I) have one or more asymmetric centers and thus can exist as enantiomers or diastereomers. Therefore, the pharmaceutical dosage forms (e.g. the solid dosage forms) according to the present invention may comprise either one or more separate individual isomers (such as the L-form and the D-form) or mixtures of isomers, preferably racemic mixtures.

The term “enantiomer”, as used herein, denotes a compound having a centre of chirality and being one of two stereoisomers that are non-superposable complete mirror images of each other. As known in the art, enantiomers differ from each other in their ability to rotate plane-polarized light and may be classified according to the CIP (Cahn-Ingold-Prelog)-convention as S- or R-enantiomer. The S- and R-configurations represent the three-dimensional orientation of the four substituents about the chiral center carbon atom.

Accordingly, an S-enantiomer of a tri-substituted glycerol compound of formula (I) has a centre of chirality at the second or middle carbon atom of the glycerol principal structure which is classified as “S” according to the CIP-convention. Such an S-enantiomer of the tri-substituted glycerol compound of formula (I) is specifically disclosed in formula (Ia) and may be used for any of the uses and in any embodiment described herein for formula (I).

In one embodiment, the tri-substituted glycerol compound of formula (I) is an S-enantiomer of a tri-substituted glycerol compound according to formula (Ia)

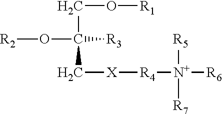

wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅, R₆ and R₇ are independently selected from the group consisting of hydrogen and methyl.

In one embodiment of the present invention, the tri-substituted glycerol compound has the formula (Ia), wherein X is phosphate, R₁ is —(CH₂)₁₇—CH₃, R₂ is CH₃, R₃ is H, R₄ is —(CH₂)₂—, and R₅, R₆ and R₇ are CH₃. Hence, in this embodiment, the tri-substituted glycerol compound of formula (Ia) is S-edelfosine or S-1-O-octadecyl-2-O-methyl-sn-glycero-3-phosphocholine or S-AP-121.

In some embodiments of the invention, the tri-substituted glycerol compounds of formula (I) are present as pharmaceutically acceptable salts. Such salts may comprise any pharmaceutically acceptable anion “neutralizing” the positive charge of the nitrogen (e.g. chloride, bromide or iodide) or any pharmaceutically acceptable cation “neutralizing” the negative charge of the phosphate or sulfate moiety (e.g. sodium or potassium cations).

In one embodiment, the present invention relates to a tri-substituted glycerol compound according to formula (I) as described herein for use in the treatment of clinically isolated syndrome, relapsing remitting multiple sclerosis (RR-MS) and/or secondary progressive multiple sclerosis (SP-MS), wherein X is phosphate, R₁ is —(CH₂)₁₇—CH₃, R₂ is CH₃, R₃ is H, R₄ is —(CH₂)₂—, and R₅ is CH₃.

In a further embodiment, the present invention relates to a tri-substituted glycerol compound according to formula (I) as described herein for use in the treatment of clinically isolated syndrome, wherein X is phosphate, R₁ is —(CH₂)₁₇—CH₃, R₂ is CH₃, R₃ is H, R₄ is —(CH₂)₂—, and R₅ is CH₃.

In another embodiment, the present invention relates to a tri-substituted glycerol compound according to formula (I) as described herein for use in the treatment of relapsing remitting multiple sclerosis (RR-MS), wherein X is phosphate, R₁ is —(CH₂)₁₇—CH₃, R₂ is CH₃, R₃ is H, R₄ is —(CH₂)₂—, and R₅ is CH₃.

In one embodiment, the present invention relates to a tri-substituted glycerol compound according to formula (I) as described herein for use in the treatment of secondary progressive multiple sclerosis (SP-MS), wherein X is phosphate, R₁ is —(CH₂)₁₇—CH₃, R₂ is CH₃, R₃ is H, R₄ is —(CH₂)₂—, and R₅ is CH₃.

In another embodiment, the present invention relates to a tri-substituted glycerol compound according to formula (I) as described herein for use in the treatment of multiple sclerosis patients not adequately responding to interferon therapy, wherein X is phosphate, R₁ is —(CH₂)₁₇—CH₃, R₂ is CH₃, R₃ is H, R₄ is —(CH₂)₂—, and R₅ is CH₃.

For use in the treatment of clinically isolated syndrome, relapsing remitting multiple sclerosis (RR-MS) and/or secondary progressive multiple sclerosis (SP-MS), the tri-substituted glycerol compound according to formula (I) as described herein may be administered in an amount of 10-40 mg/day, optionally 20-30 mg/day. Exemplary doses include 10-15 mg/day or 10-20 mg/day or 10-25 mg/day or 15-25 mg/day.

For use in the treatment of multiple sclerosis patients not adequately responding to interferon therapy, the tri-substituted glycerol compound according to formula (I) as described herein may be administered in an amount of 10-40 mg/day, optionally 20-30 mg/day. Exemplary doses include 10-15 mg/day or 10-20 mg/day or 10-25 mg/day or 15-25 mg/day.

The daily dosage of the tri-substituted glycerol compound may be administered as a single dose or in multiple doses such as two or three individual doses administered during the day, e.g. in the morning, at noon, and at night.

In some embodiments, the tri-substituted compound according to formula (I) as described herein may be administered via any parenteral or non-parenteral route. Parenteral application methods comprise, for example, intracutaneous, subcutaneous, intramuscular or intravenous injection and infusion techniques. Non-parenteral delivery modes include, for instance, oral or topical administration. Furthermore, the tri-substituted glycerol compound according to formula (I) as described herein as well as the further pharmaceutically active compound(s) may be administered locally or systemically. In one embodiment of the invention the tri-substituted compound according to formula (I) as defined herein is administered orally.

The tri-substituted glycerol compound according to formula (I) for use as described herein may be administered alone or in combination with at least one further pharmaceutically active compound. In case a combination of compounds or active agents is used, the individual compound or active ingredients may be administered via the same or via different administration routes. The further pharmaceutically active compound may be administered via any administration route mentioned above for the tri-substituted glycerol compound according to the present invention. In one embodiment of the invention, the tri-substituted glycerol compound according to formula (I) as described herein and the at least one further pharmaceutically active compound are administered orally.

The tri-substituted glycerol compound according to formula (I) for use as described herein and the at least one further pharmaceutically active compound may be administered at the same time or at different time points. The tri-substituted compound according to formula (I) as described herein may be administered first, followed by the administration of the at least one pharmaceutically active compound or vice versa. Any sequence of administration of the tri-substituted compound as described herein and the at least one further pharmaceutically active compound is encompassed in the present invention, e.g. the tri-substituted glycerol compound according to formula (I) as described herein may also be administered in between two further pharmaceutically active compounds.

Pharmaceutically active compounds which may be administered in combination with the tri-substituted glycerol compound according to formula (I) for use as described herein include compounds labelled for use in the treatment of multiple sclerosis, compounds currently studied in clinical trials for the treatment of multiple sclerosis and/or any other compound which is suitable for the treatment of multiple sclerosis. A non-exhaustive list of such compounds includes glucocorticoids, interferone-beta compounds, immunosuppressive agents, immunomodulatory compounds, monoclonal antibodies, intravenous immunoglobulin and oral fumarate.

Examples of glucocorticoids within the meaning of the present invention include cortisone, prednisone, prednisolone and methyl-prednisolone. Such glucocorticoids are commonly applied e.g. during acute attacks.

Further compounds which could be used in the treatment of multiple sclerosis are interferone-beta compounds, which are frequently used in the treatment of relapsing-remitting multiple sclerosis. Interferone-beta compounds within the meaning of the present invention include interferone-beta 1a compounds such as the compounds sold under the trade names Avonex, CinnoVex, ReciGen and Rebfib as well as interferone-beta 1b compounds such as the compounds sold under the trade names Betaferone, Betaseron and Extavia.

Immunosuppressive agents may also be used for treatment of multiple sclerosis. Exemplary immunosuppressive agents which can be used in combination with the tri-substituted glycerol compounds of formula (I) or (Ia) as described herein include mitoxantrone, cyclophosphamid, methotrexat, azathioprine, mycophenolate, cladribine and fingolimod.

Immunomodulatory compounds within the meaning of the present invention include the non-steroid immunomodulatory compound glatiramer acetate (GA), the synthetic peptide dirucotide, the oral immunomodulatory drug laquinimod and teriflunomid having anti-proliferative/anti-inflammatory properties.

Monoclonal antibodies such as natalizumab, daclizumab, alemtuzumab and rituximab may also be used in combination with the tri-substituted glycerol compounds according to formula (I) or (Ia) as described herein.

A further pharmaceutically active compound which may be used in combination with the tri-substituted glycerol compounds of formula (I) or (Ia) as described herein is intravenous immunoglobulin (IVG).

Furthermore, oral fumarate (BG00012) may also be used in combination with the tri-substituted glycerol compounds according to formula (I) or (Ia) as described herein.

Compounds including inosine, Neurovax, Tovaxin, ATL1102, CDP323, estradiol and estrogen receptors, Ibudilast, Inosine, Ocrelizumab, Ofatumumab, BAF312 and BCG vaccine may also be used in combination with the tri-substituted glycerol compounds according to formula (I) or (Ia) as described herein.

Other compounds which may be suitable to be administered together with the tri-substituted glycerol compounds of formula (I) or (Ia) for uses described herein include antimicrobial agents against Chlamydophila pneumonia, antioxidants, Bilirubin, Hydralazine, helminthic therapy, low dose naltrexone, minocycline, pixantrone, prolactins, statins, testosterone, vitamin D and/or omega-3 fatty acids.

It is understood within the meaning of the present invention that each compound mentioned above may be used in combination with the tri-substituted compounds according to formula (I) or (Ia) as described herein either alone or in combination with a further compound mentioned herein.

In some embodiments of the invention the tri-substituted glycerol compound according to formula (I) or (Ia) as described herein is used in combination with glucocorticoids. In some embodiments of the invention the tri-substituted glycerol compound according to formula (I) or (Ia) as described herein is used in combination with interferone-beta compounds. In some embodiments of the invention the tri-substituted glycerol compound according to formula (I) or (Ia) as described herein is used in combination with immunosuppressive agents. In some embodiments of the invention the tri-substituted glycerol compound according to formula (I) or (Ia) as described herein is used in combination with immunomodulatory compounds. In some embodiments of the invention the tri-substituted glycerol compound according to formula (I) or (Ia) as described herein is used in combination with monoclonal antibodies.

In some embodiments of the invention the tri-substituted glycerol compound according to formula (I) as described herein is used in combination with a compound having a different mode of action than the tri-substituted glycerol compounds according to the invention, in particular than edelfosine.

In some embodiments of the invention the tri-substituted glycerol compound according to formula (I) or (Ia) as described herein is used in combination with a compound selected from the group consisting of teriflunomid, laquinimod and oral fumarate. In some embodiments of the invention the tri-substituted glycerol compound according to formula (I) or (Ia) as described herein is used in combination with teriflunomid. In some embodiments of the invention the tri-substituted glycerol compound according to formula (I) or (Ia) as described herein is used in combination with laquimimod. In some embodiments of the invention the tri-substituted glycerol compound according to formula (I) or (Ia) as described herein is used in combination with oral fumarate. In some embodiments of the invention the tri-substituted glycerol compound according to formula (I) or (Ia) as described herein is used in combination with fingolimod.

Another aspect of the invention relates to a tri-substituted compound according to formula (I)

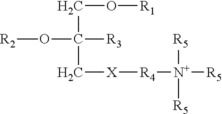

or an enantiomer or diastereomer or a pharmaceutically acceptable salt thereof for use as a medicament, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl,
wherein the tri-substituted glycerol compound is administered in combination with at least one further pharmaceutically active compound as defined herein.

Pharmaceutically active compounds which may be administered in combination with the tri-substituted glycerol compound according to formula (I) for use as a medicament include compounds labelled for use in the treatment of multiple sclerosis, compounds currently studied in clinical trials for the treatment of multiple sclerosis and/or any other compound which is suitable for the treatment of multiple sclerosis. A non-exhaustive list of such compounds includes glucocorticoids, interferone-beta compounds, immunosuppressive agents, immunomodulatory compounds, monoclonal antibodies, intravenous immunoglobulin and oral fumarate.

Examples of glucocorticoids which can be used in combination with the tri-substituted glycerol compounds of formula (I) or (Ia) include cortisone, prednisone, prednisolone and methyl-prednisolone. Such glucocorticoids are commonly applied e.g. during acute attacks.

Further compounds which could be used in the treatment of multiple sclerosis are interferone-beta compounds, which are frequently used in the treatment of relapsing-remitting multiple sclerosis. Interferone-beta compounds which can be used in combination with the tri-substituted glycerol compounds of formula (I) or (Ia) include interferone-beta 1a compounds such as the compounds sold under the trade names Avonex, CinnoVex, ReciGen and Rebfib as well as interferone-beta 1b compounds such as the compounds sold under the trade names Betaferone, Betaseron and Extavia.

Immunosuppressive agents may also be used for treatment of multiple sclerosis. Exemplary immunosuppressive agents which can be used in combination with the tri-substituted glycerol compounds of formula (I) or (Ia) as described herein include mitoxantrone, cyclophosphamid, methotrexat, azathioprine, mycophenolate, cladribine and fingolimod.

Immunomodulatory compounds within the meaning of the present invention include the non-steroid immunomodulatory compound glatiramer acetate (GA), the synthetic peptide dirucotide, the oral immunomodulatory drug laquinimod and teriflunomid having anti-proliferative/anti-inflammatory properties.

Monoclonal antibodies such as natalizumab, daclizumab, alemtuzumab and rituximab may also be used in combination with the tri-substituted glycerol compounds according to formula (I) or (Ia) as described herein.

A further pharmaceutically active compound which may be used in combination with the tri-substituted glycerol compounds of formula (I) or (Ia) as described herein is intravenous immunoglobulin (IVG).

Furthermore, oral fumarate (BG00012) may also be used in combination with the tri-substituted glycerol compounds according to formula (I) or (Ia) as described herein.

Compounds presently tested in clinical trials include inosine, Neurovax, Tovaxin, ATL1102, CDP323, estradiol and estrogen receptors, Ibudilast, Inosine, Ocrelizumab, Ofatumumab, BAF312 and BCG vaccine.

Other compounds which may be suitable to be administered together with the tri-substituted glycerol compounds of formula (I) or (Ia) for uses as a medicament include antimicrobial agents against Chlamydophila pneumonia, antioxidants, Bilirubin, Hydralazine, helminthic therapy, low dose naltrexone, minocycline, pixantrone, prolactins, statins, testosterone, vitamin D and omega-3 fatty acids.

It is understood within the meaning of the present invention that each compound mentioned above may be used in combination with the tri-substituted compounds according to formula (I) or (Ia) as described herein either alone or in combination with a further compound mentioned herein.

In some embodiments of the invention the tri-substituted glycerol compound according to formula (I) or (Ia) as described herein is used in combination with glucocorticoids. In some embodiments of the invention the tri-substituted glycerol compound according to formula (I) or (Ia) as described herein is used in combination with interferone-beta compounds. In some embodiments of the invention the tri-substituted glycerol compound according to formula (I) or (Ia) as described herein is used in combination with immunosuppressive agents. In some embodiments of the invention the tri-substituted glycerol compound according to formula (I) or (Ia) as described herein is used in combination with immunomodulatory compounds. In some embodiments of the invention the tri-substituted glycerol compound according to formula (I) or (Ia) as described herein is used in combination with monoclonal antibodies.

In some embodiments of the invention the tri-substituted glycerol compound according to formula (I) or (Ia) as described herein is used in combination with a compound having a different mode of action than the tri-substituted glycerol compounds according to the invention, in particular than edelfosine.

In some embodiments of the invention the tri-substituted glycerol compound according to formula (I) or (Ia) as described herein is used in combination with a compound selected from the group consisting of teriflunomid, laquinimod and oral fumarate. In some embodiments of the invention the tri-substituted glycerol compound according to formula (I) or (Ia) as described herein is used in combination with teriflunomid. In some embodiments of the invention the tri-substituted glycerol compound according to formula (I) or (Ia) as described herein is used in combination with laquimimod. In some embodiments of the invention the tri-substituted glycerol compound according to formula (I) or (Ia) as described herein is used in combination with oral fumarate. In some embodiments of the invention the tri-substituted glycerol compound according to formula (I) or (Ia) as described herein is used in combination with fingolimod.

In one embodiment the tri-substituted glycerol compound according to formula (I) for use as a medicament, which is administered in combination with at least one further pharmaceutically active compound as described above, is a compound according to the above formula (I), wherein X is phosphate, R₁ is —(CH₂)₁₇—CH₃, R₂ is CH₃, R₃ is H, R₄ is —(CH₂)₂—, and R₅ is CH₃ or S-edelfosine.

In some embodiments the tri-substituted glycerol compound according to formula (I) as described herein for use as a medicament, which is administered in combination with at least one further pharmaceutically active compound as described above is administered orally.

In one embodiment, the tri-substituted compound according to formula (I) or (Ia) for any of the uses as defined is administered as a pharmaceutical dosage form suitable for oral application. In particular, the dosage form may be a solid dosage form. Examples of such dosage forms include inter alia tablets, pills, capsules, granulates, pellets, powders, multi-particulate formulations (e.g., beads, granules or crystals), and dragees. The unit doses of multi-particulates may be incorporated into a solid dosage form, e.g. via compression or shaping into tablets or by placing a requisite amount inside a gelatin capsule.

All these solid dosage forms for oral application as well as methods for their preparation are well established in the art (see, e.g., Gennaro, A. L. and Gennaro, A. R. (2000) Remington: The Science and Practice of Pharmacy, 20th Ed., Lippincott Williams & Wilkins, Philadelphia, Pa.; Ritschel, W. A. & Bauer-Brandl, A. (2002) Die Tablette: Handbuch der Entwicklung, Herstellung and Qualitätssicherung. Editio-Cantor Verlag, Aulendorf, Germany; Crowder, T. M. et al. (2003) A Guide to Pharmaceutical Particulate Science. Interpharm/CRC, Boca Raton, Fla.; Stricker, H. (2003) Arzneiformenentwicklung, Springer Verlag, Berlin, Germany; Niazi, S. K. (2004) Handbook of Pharmaceutical Manufacturing Formulations, CRC Press, Boca Raton, Fla.).

In exemplary embodiments of the invention, the pharmaceutical solid dosage form is selected from the group consisting of tablets, pills, capsules, granulates, pellets, powders, multi-particulate formulations, dragees and granules. In particular, the pharmaceutical solid dosage form may be selected from the group consisting of tablets, pills, capsules and granules.

Pharmaceutical compositions comprising tri-substituted glycerol compounds for oral administration are exemplified in WO 2008/055996.

In one embodiment of the invention the pharmaceutical dosage form described herein may further comprise a pharmaceutically acceptable excipient. The term “pharmaceutically acceptable excipient” in the meaning of the present invention can be any substance used for the preparation of pharmaceutical dosage forms such as coating materials, film-forming materials, fillers, disintegrating agents, release-modifying materials, carrier materials, diluents, binding agents and other adjuvants.

A further embodiment relates to the tri-substituted glycerol according to formula (I) compound as defined above for use as a medicament, wherein the medicament is for the treatment of multiple sclerosis and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of clinically isolated syndrome, relapsing remitting multiple sclerosis (RR-MS), secondary progressive multiple sclerosis (SP-MS) and/or multiple sclerosis patients not adequately responding to interferon therapy and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of clinically isolated syndrome, relapsing remitting multiple sclerosis (RR-MS) and/or secondary progressive multiple sclerosis (SP-MS) and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of clinically isolated syndrome, and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of secondary progressive multiple sclerosis (SP-MS) and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of relapsing remitting multiple sclerosis (RR-MS), and wherein the tri-substituted glycerol compound is administered in combination with at least one further pharmaceutically active compound as defined above.

Another embodiment of the invention relates to the tri-substituted glycerol compound according to formula (I) or (Ia) as defined above for use as a medicament, wherein the medicament is for the treatment of multiple sclerosis patients not adequately responding to interferon therapy and wherein the tri-substituted glycerol compound is administered in combination with at least one further pharmaceutically active compound as defined above.

Where the term “comprise” or “comprising” is used in the present description and claims, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

All documents cited or referenced herein including any manufacturer's instructions, descriptions, product specifications, and product sheets for any products mentioned herein or in any document referenced herein, are hereby incorporated by reference and may be employed in the practice of the invention. Citation or identification of any document in this application is not an admission that such document is available as prior art to the present invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%.

The invention is further described in the following examples which are solely for the purpose of illustrating specific embodiments of the invention, and are also not to be construed as limiting the scope of the invention in any way.

### EXAMPLES

### Example 1

### Edelfosine Interferes with Proliferation of Human PBMCs after Mitogenic Activation, but Also with Proliferation of Antigen-Specific T-Cell Lines

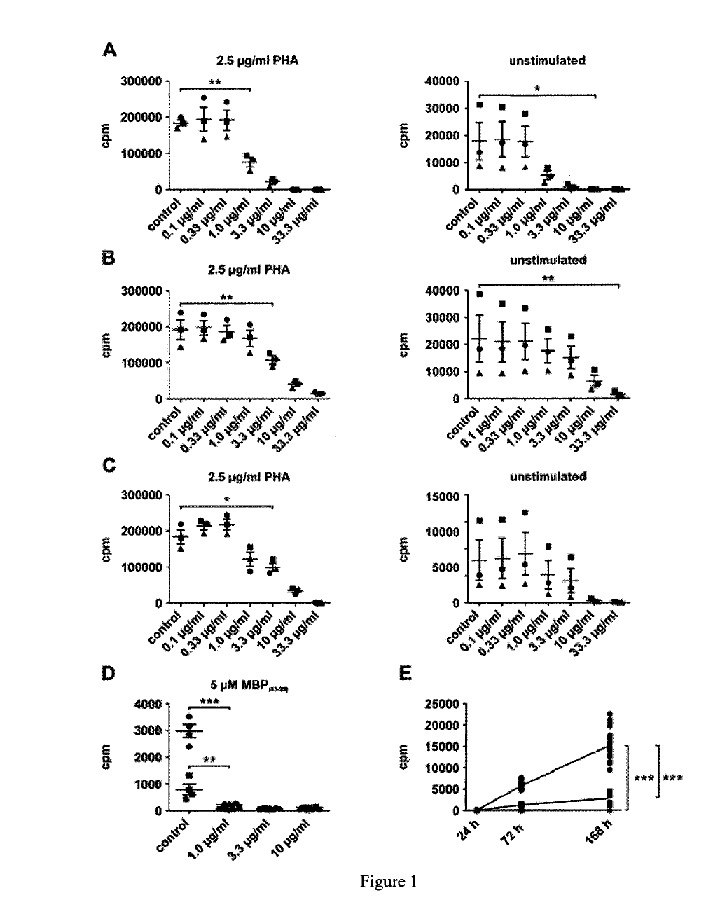

In order to characterize the influence of edelfosine on the proliferation of human T cells, peripheral blood mononuclear cells (PBMCs) were first stimulated with a mitogenic stimulus, PHA (phytohemagglutinin) Proliferation was analyzed after three days of culture. First, the impact of edelfosine on T-cell activation and proliferation was investigated by adding edelfosine immediately as the cells were seeded. Cells were incubated without or in the presence of PHA. A reduction in proliferation was already detectable upon addition of 1.0 μg/ml edelfosine and also found in the case of higher concentrations (FIG. 1A). Unstimulated controls also showed a reduction in cellular proliferation in presence of 10 μg/ml edelfosine or higher concentrations. Here, the half maximal inhibitory concentration (IC₅₀) was determined by nonlinear regression. For stimulated cells the IC₅₀ was 0.64±0.10 μg/ml edelfosine. In the absence of PHA the IC₅₀ was 0.44±0.05 μg/ml edelfosine.

The second approach aimed at characterizing the edelfosine influence on already proliferating cells. For that reason cells were activated with PHA, and edelfosine was added two days later. Here, concentrations of 3.3 μg/ml edelfosine or higher resulted in an efficient reduction of proliferation (FIG. 1B). In comparison, 33.3 μg/ml edelfosine was found to significantly reduce the proliferation in unstimulated controls.

To determine whether edelfosine, which was found to affect viability of unstimulated cells, may also compromise the capacity of T cells to proliferate upon stimulation PBMCs were incubated with or without edelfosine for 24 h in the absence of stimulation. Cells were washed extensively to remove edelfosine followed by further culturing for three days. In control approaches cells were incubated without PHA. Cells were found to retain their proliferative function after incubation with edelfosine at concentrations of up to 1 μg/ml (FIG. 1C). 3.3 μg/ml edelfosine or higher interfered with the cellular capacity to proliferate upon stimulation with PHA. With regard to unstimulated cells, no significant reductions of proliferation were identified.

To further investigate the influence of edelfosine on T-cell proliferation in the context of antigen-specific stimulation, T-cell lines (TCLs) specific for MBP₍₈₃₋₉₉₎ were used. Edelfosine was added immediately after seeding of cells. After three days of culture 1.0 μg/ml edelfosine was already detected to profoundly reduce T-cell proliferation in stimulated as well as unstimulated conditions (FIG. 1D).

In an additional approach PBMCs were incubated for up to seven days without the addition of a stimulus. Cells were cultured in medium alone, in presence of anti-HLA-DR- and anti-MHC class I-blocking antibody or with 3.3 μg/ml edelfosine. Both the addition of the antibody and edelfosine resulted in considerably lower proliferations. In the case of edelfosine the proliferation was reduced to non-detectable levels (FIG. 1E). These results implied an effect of edelfosine on cellular proliferation even if cells were not activated by adding a defined stimulus, i.e. in the unstimulated condition.

### Example 2

### Whole Genome Expression Analysis of CD4+ T Cells Reveals Impact of Edelfosine on a Distinct Set of Signaling Pathways

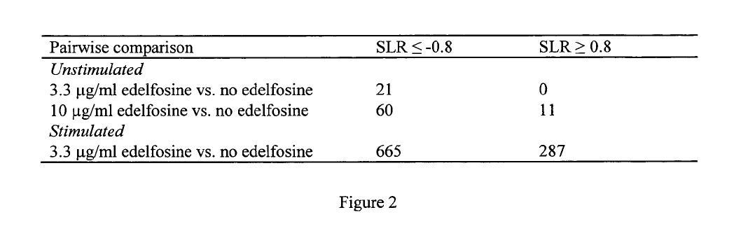

Enriched CD4+ T cells from PBMCs of two age-matched female as well as two age-matched male donors were incubated for 30 h without edelfosine, in presence of 3.3 μg/ml edelfosine and 10 μg/ml edelfosine, respectively. In parallel approaches, cells were incubated with beads coated with antibodies against CD2, CD3 and CD28 or with coated beads and 3.3 μg/ml edelfosine. Cell-culture supernatant was saved and cells were subjected to RNA isolation, cDNA synthesis and microarray analysis for gene expression. Comparative gene-expression analysis was performed according to FIG. 2.

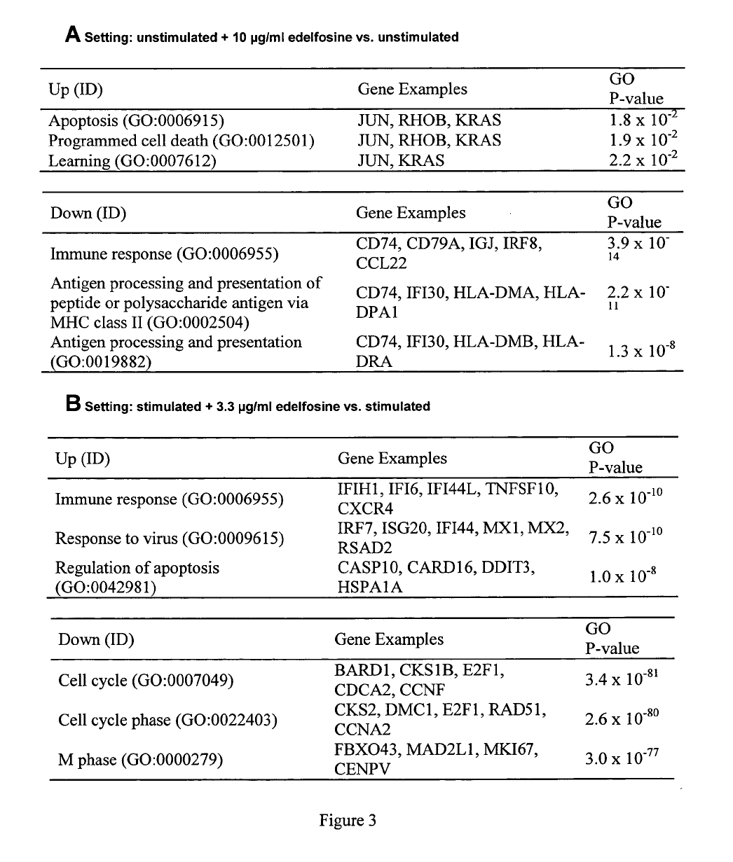

In general, edelfosine modulated the gene expression of human CD4+ T cells in the case of stimulation but also if no exogenous stimulus was added, although to a limited extent. Except for the transcription factor 4 (TCF4) gene, every significantly downregulated gene in cells cultured with 3.3 μg/ml edelfosine could also be identified in cells cultured with 10 μg/ml edelfosine. Moreover, the latter condition yielded 11 upregulated genes (SLR≧0.8) and additional downregulated genes (SLR≦−0.8). Therefore, further analysis of the unstimulated cells focused on the 10 μg/ml edelfosine approach. Next, the database for annotation, visualization and integrated discovery (DAVID)-bioinformatics database was used to annotate the differentially expressed genes to functional themes/groups and assign to them so-called gene-ontology (GO) terms for specific pathways of cellular function ₍₃₁₄₎. The GO project is an effort to consistently describe gene products in databases. DAVID, as a tool, is designed to allow systematical mapping of large numbers of genes in lists associated with biological annotations (e.g. GO terms). Three structured controlled vocabularies (ontologies) have been developed to describe gene products in a species-independent manner: the associated biological process (BP), cellular components and molecular functions. The GOTERM_BP_FAT set which has been specifically designed by the DAVID consortium was selected to filter the broadest terms in order to prevent overshadowing of more specific terms. FIG. 3 summarizes the top-3 GO-terms which were selected due to their highest GO P-values. As shown therein, the addition of 10 mg/kg edelfosine was leading to an upregulation of genes associated with apoptosis and cell death in the unstimulated setting, a finding that was in accordance with the previously described analysis for annexin V and PI after edelfosine incubation.

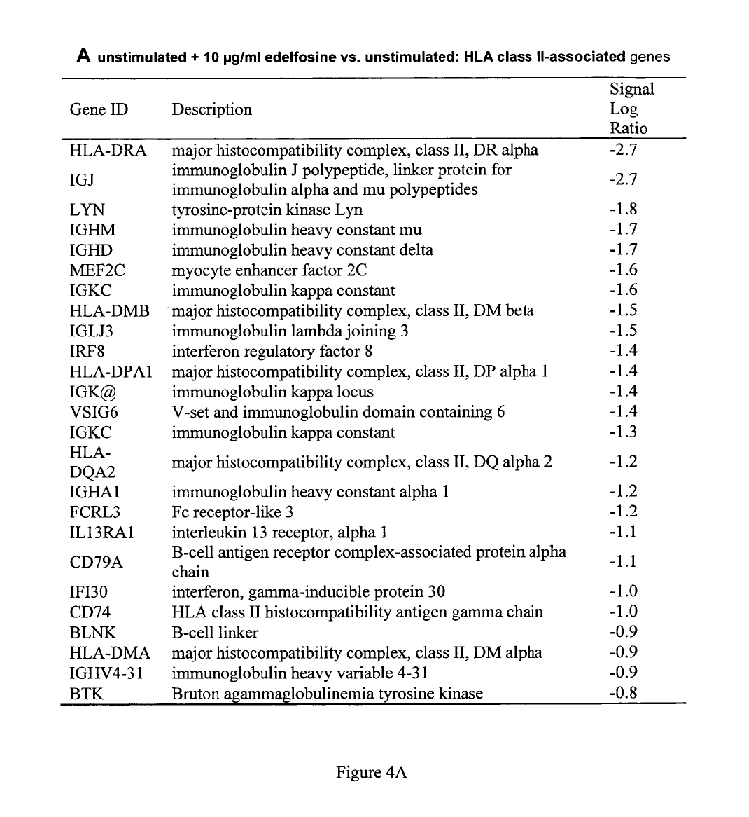

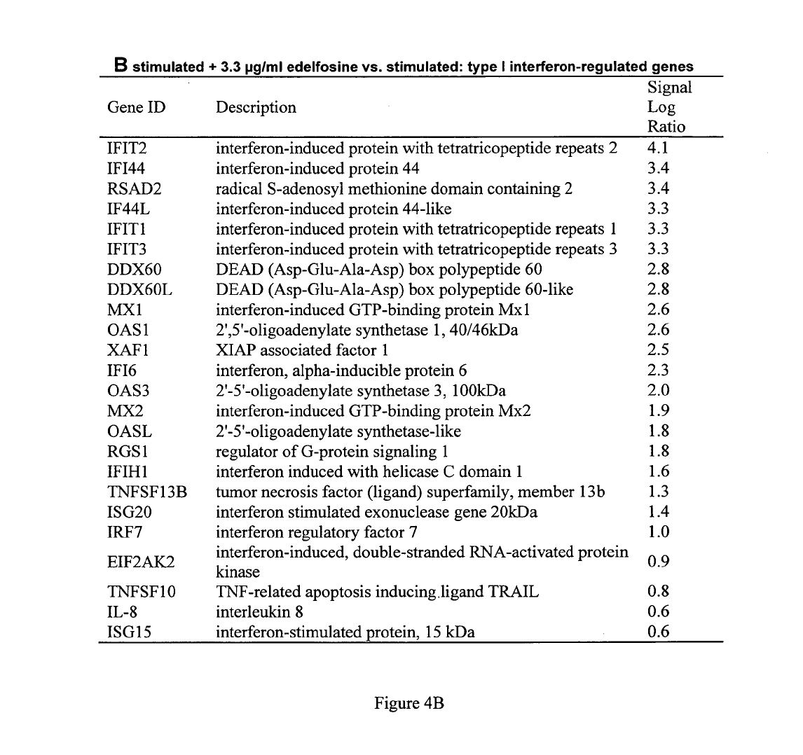

A second finding was the downmodulation of MHC class II-associated genes, if cells were cultured in presence of 10 μg/ml edelfosine without stimulus. Here, genes were clustered by the GO-terms immune response, antigen processing and presentation of peptide or polysaccharide antigen via MHC class II, or antigen processing and presentation. In stimulated CD4+ T cells edelfosine interfered with the cell-cycle progression by downmodulation of cell cycle-associated genes. Strikingly, besides the upregulation of genes associated with the regulation of apoptosis, the incubation of stimulated cells with 3.3 μg/ml edelfosine was leading to the upregulation of genes that were allocated to GO-terms like immune response and response to virus. Most of these genes were found to be type I interferon-associated genes. Gene lists were created to focus especially on the prominent, novel identified biological pathways that appeared to be modulated upon edelfosine treatment of human CD4+ T cells (FIG. 4).

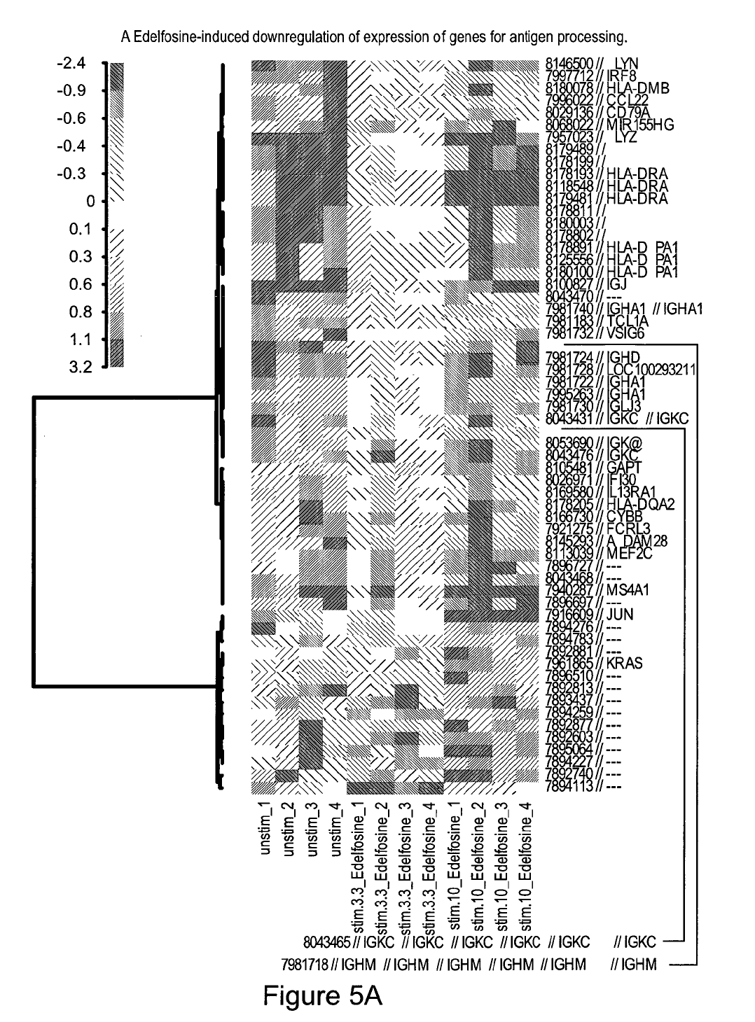

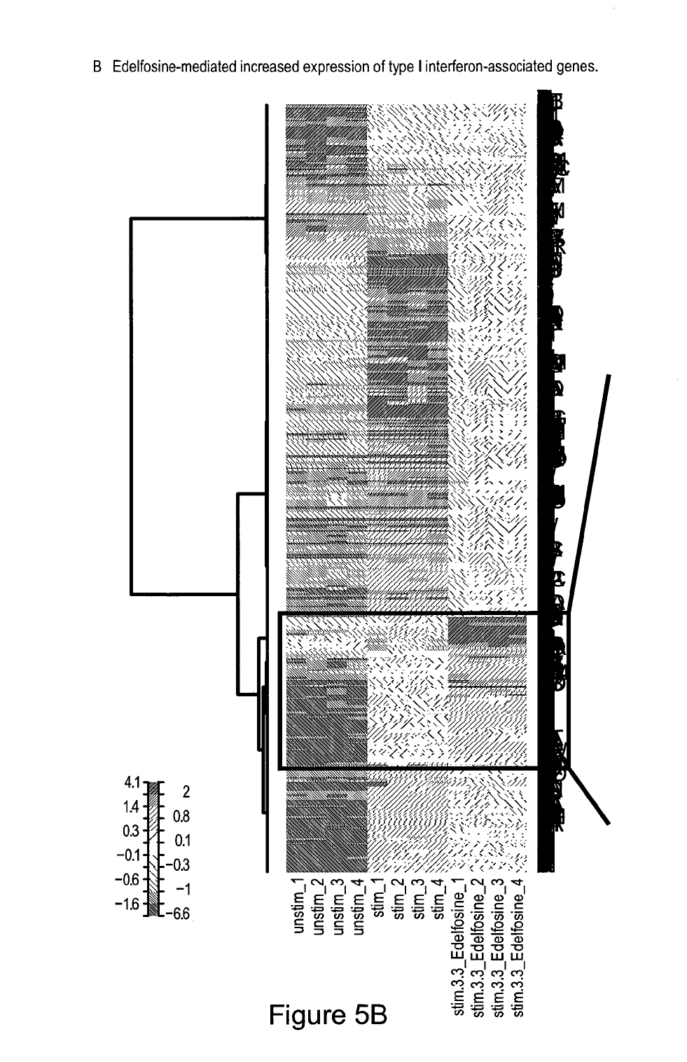

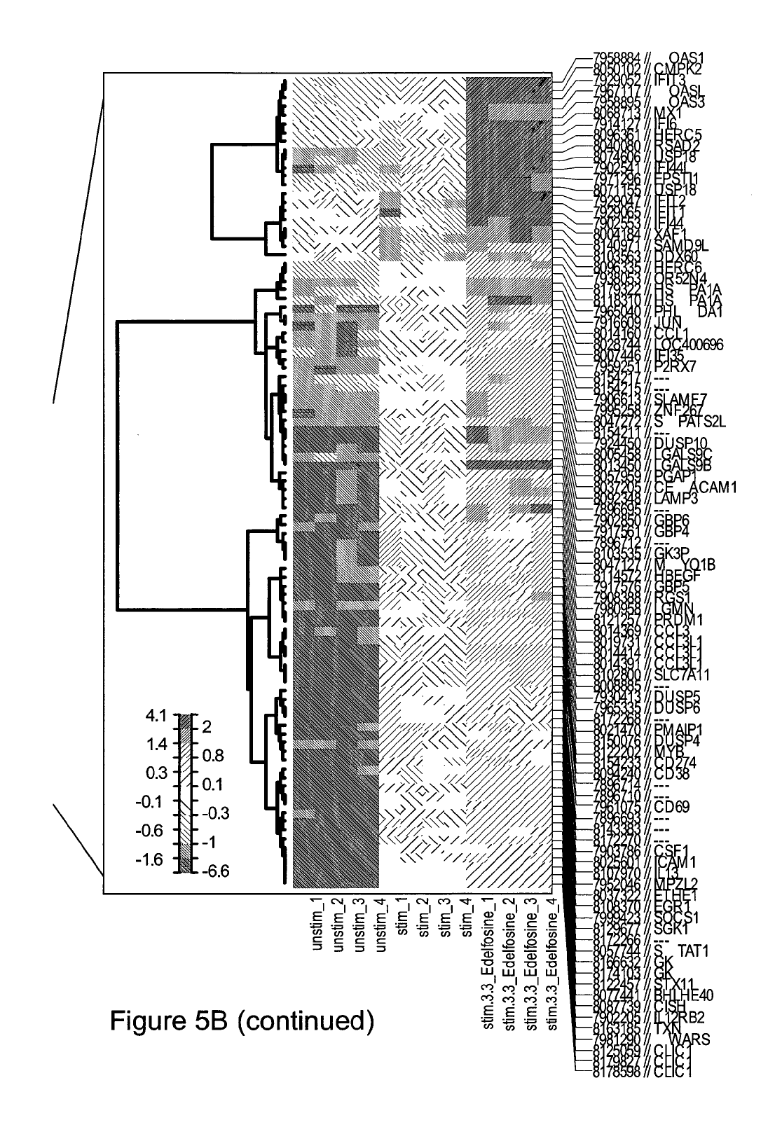

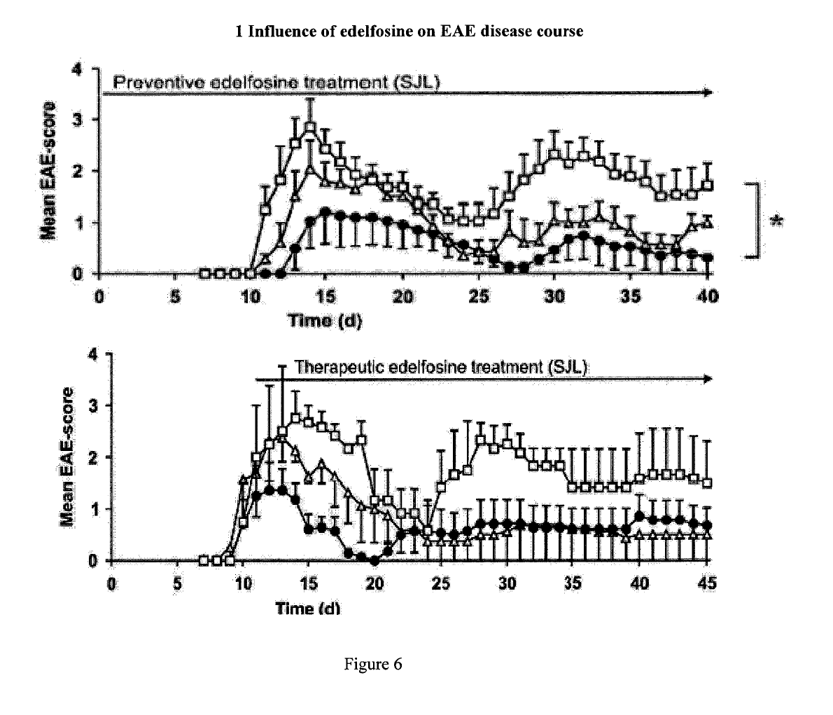

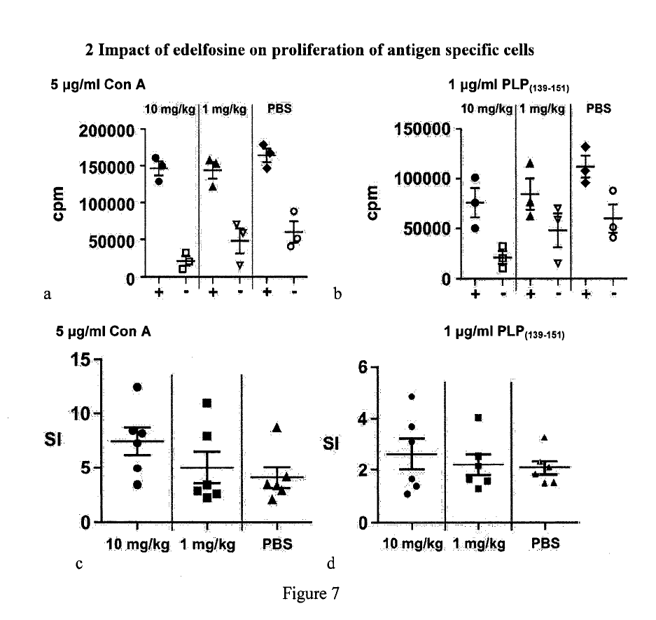

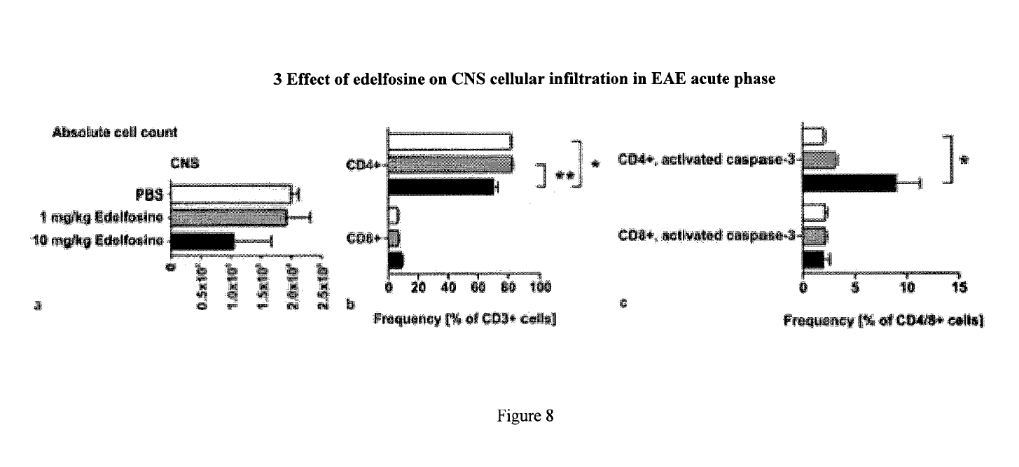

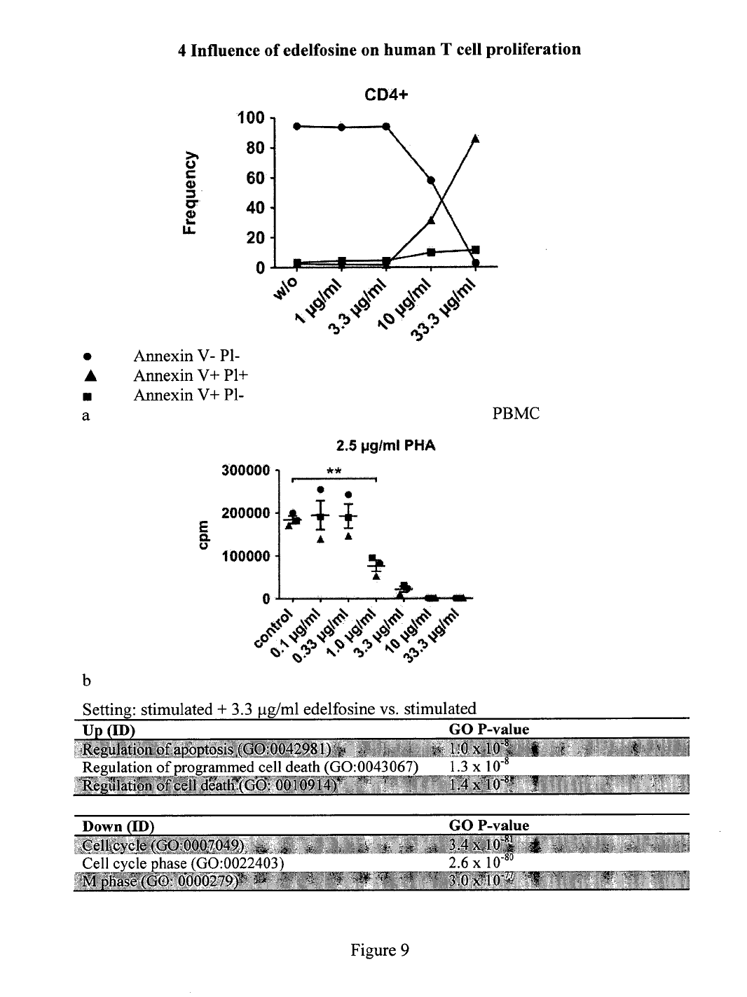

In the case of unstimulated cells that were incubated with 10 μg/ml edelfosine, the list of downmodulated genes summarizes genes with SLR≦−0.8 which could be allocated to the GO terms immune response and antigen processing and presentation. These genes were largely assignable to MHC class II- and immunoglobulin-regulation. The second gene list collects genes that appeared to be upregulated in human stimulated CD4+ T cells if cultured with 3.3 μg/ml edelfosine. This list contains not only genes allocated to type I interferon-associated immune responses with SLR≧0.8 but also type I interferon-associated genes with SLR=0.6 that were previously detected in a longitudinal gene-expression study of PBMCs derived from interferon beta-treated MS patients ₍₃₁₅₎. In order to determine and to visualize patterns of gene expression consecutive comparisons were performed by generating heatmaps (FIG. 5). The first sequence for downregulated, antigen processing- and presentation-related genes was as follows: approach “unstimulated” in comparison to approaches “unstimulated with 3.3₁1.1 g/ml edelfosine” and “unstimulated with 10 μg/ml edelfosine”. The heatmap indicates the consecutive downregulation of genes with increasing edelfosine concentration. Only those genes were considered that presented with at least one SLR≦−0.8 in all three possible pairwise comparisons. Thus, in absence of edelfosine the highest expression levels were determined for these genes that may be involved in antigen-processing and presentation. The second sequence comprised upregulated, immune and virus response-allocated genes by comparing the “unstimulated” condition to the approaches “stimulated” and “stimulated with 3.3 μg/ml edelfosine”. Here, only those genes were considered that presented with at least two SLR≧0.8 in all three possible pairwise comparisons.

The constructed heatmap emphasized the consistent upregulation of immune and virus response-associated, type I interferon-related genes upon stimulation in presence of 3.3 μg/ml edelfosine. Provided that cells were incubated without edelfosine, the absence and presence of a stimulus for CD4+ T cells resulted in the clustering of immune- and virus-allocated genes into two subgroups. These groups were either characterized by a subtle upregulation of genes if cells were stimulated or downregulation in case of stimulation. Despite these detected differences in gene expression between the two clusters, they could not be discriminated by divergent induction: genes in both clusters are interferon-inducible. Regardless of this clustering of genes in the absence of edelfosine, the addition of 3.3 μg/ml edelfosine induced a consistent and profound upregulation of expression of these genes.

### REFERENCES USED IN THE EXAMPLES

83. M. Pette et al., Myelin basic protein-specific T lymphocyte lines from MS patients and healthy individuals, Neurology 40, 1770-6 (1990).
84. J. R. Richert, D. E. McFarlin, J. W. Rose, H. F. McFarland, J. I. Greenstein, Expansion of antigen-specific T cells from cerebrospinal fluid of patients with multiple sclerosis, Journal of Neuroimmunology 5, 317-24 (1983).
85. C. B. Pettinelli, R. B. Fritz, C. H. Chou, D. E. McFarlin, Encephalitogenic activity of guinea pig myelin basic protein in the SJL mouse, Journal of Immunology 129, 1209-11 (1982).
86. R. Martin, H. F. McFarland, D. E. McFarlin, Immunological aspects of demyelinating diseases, Annual Review of Immunology 10, 153-87 (1992).
87. S. S. Zamvil, L. Steinman, The T lymphocyte in experimental allergic encephalomyelitis, Annual Review of Immunology 8, 579-621 (1990).
88. C. B. Pettinelli, D. E. McFarlin, Adoptive transfer of experimental allergic encephalomyelitis in SJL/J mice after in vitro activation of lymph node cells by myelin basic protein: requirement for Lyt 1+ 2− T lymphocytes, Journal of Immunology 127, 1420-3 (1981).
89. B. Pöllinger et al., Spontaneous relapsing-remitting EAE in the SJL/J mouse: MOG-reactive transgenic T cells recruit endogenous MOG-specific B cells, The Journal of Experimental Medicine 206, 1303-16 (2009).
90. J. Goverman et al., Transgenic mice that express a myelin basic protein-specific T cell receptor develop spontaneous autoimmunity, Cell 72, 551-60 (1993).
91. T. G. Forsthuber et al., T cell epitopes of human myelin oligodendrocyte glycoprotein identified in HLA-DR4 (DRB1*0401) transgenic mice are encephalitogenic and are presented by human B cells, Journal of Immunology 167, 7119-25 (2001).
92. K. Kawamura et al., Hla-DR2-restricted responses to proteolipid protein 95-116 peptide cause autoimmune encephalitis in transgenic mice, The Journal of Clinical Investigation 105, 977-84 (2000).
93. P. Das et al., Complementation between specific HLA-DR and HLA-DQ genes in transgenic mice determines susceptibility to experimental autoimmune encephalomyelitis, Human Immunology 61, 279-89 (2000).
94. L. S. Madsen et al., A humanized model for multiple sclerosis using HLA-DR2 and a human T-cell receptor, Nature Genetics 23, 343-7 (1999).
95. J. A. Quandt et al., Unique clinical and pathological features in HLA-DRB1*0401-restricted MBP 111-129-specific humanized TCR transgenic mice, The Journal of Experimental Medicine 200, 223-34 (2004).
96. B. Bielekova et al., Encephalitogenic potential of the myelin basic protein peptide (amino acids 83-99) in multiple sclerosis: results of a phase II clinical trial with an altered peptide ligand, Nature Medicine 6, 1167-75 (2000).
97. B. Bielekova et al., Expansion and functional relevance of high-avidity myelin-specific CD4+ T cells in multiple sclerosis, Journal of Immunology 172, 3893-904 (2004).
98. J. M. van Noort et al., The small heat-shock protein alpha B-crystallin as candidate autoantigen in multiple sclerosis, Nature 375, 798-801 (1995).
99. S. S. Ousman et al., Protective and therapeutic role for alphaB-crystallin in autoimmune demyelination, Nature 448, 474-9 (2007).
139. C. M. Poser et al., New diagnostic criteria for multiple sclerosis: guidelines for research protocols, Annals of Neurology 13, 227-31 (1983).
140. G. A. Schumacher et al., Problems of experimental trials of therapy in multiple sclerosis: report by the panel on the evaluation of experimental trials of therapy in multiple sclerosis, Annals of the New York Academy of Sciences 122, 552-68 (1965).
141. W. I. McDonald et al., Recommended diagnostic criteria for multiple sclerosis: guidelines from the International Panel on the diagnosis of multiple sclerosis, Annals of Neurology 50, 121-7 (2001).
142. C. H. Polman et al., Diagnostic criteria for multiple sclerosis: 2010 revisions to the McDonald criteria, Annals of Neurology 69, 292-302 (2011).
143. H. F. McFarland, Examination of the role of magnetic resonance imaging in multiple sclerosis: A problem-orientated approach, Annals of Indian Academy of Neurology 12, 254-63 (2009).
144. H. F. McFarland et al., Using gadolinium-enhanced magnetic resonance imaging lesions to monitor disease activity in multiple sclerosis, Annals of Neurology 32, 758-66 (1992).
145. R. D. Davenport, D. F. Keren, Oligoclonal bands in cerebrospinal fluids: significance of corresponding bands in serum for diagnosis of multiple sclerosis, Clinical Chemistry 34, 764-5 (1988).
146. H. Brønnum-Hansen, N. Koch-Henriksen, E. Stenager, Trends in survival and cause of death in Danish patients with multiple sclerosis, Brain 127, 844-50 (2004).
147. B. Runmarker, O. Andersen, Prognostic factors in a multiple sclerosis incidence cohort with twenty-five years of follow-up, Brain 116 (Pt 1, 117-34 (1993).
148. C. Confavreux, G. Aimard, M. Devic, Course and prognosis of multiple sclerosis assessed by the computerized data processing of 349 patients, Brain 103, 281-300 (1980).
149. J. H. Noseworthy, C. Lucchinetti, M. Rodriguez, B. G. Weinshenker, Multiple sclerosis, The New England Journal of Medicine 343, 938-52 (2000).
150. H. Lassmann, W. Brück, C. F. Lucchinetti, The immunopathology of multiple sclerosis: an overview, Brain Pathology 17, 210-8 (2007).
151. A. Compston, A. Coles, Multiple sclerosis, Lancet 372, 1502-17 (2008).
314. D. W. Huang, B. T. Sherman, R. A. Lempicki, Systematic and integrative analysis of large gene lists using DAVID bioinformatics resources, Nature Protocols 4, 44-57 (2009).
315. P. Serrano-Fernandez et al., Time course transcriptomics of IFNB1b drug therapy in multiple sclerosis, Autoimmunity 43, 172-8 (2010).

1. A method of treating clinically isolated syndrome, relapsing remitting multiple sclerosis (RR-MS), and/or secondary progressive multiple sclerosis (SP-MS) in a patient comprising administering an effective amount of a tri-substituted glycerol compound of formula (I) 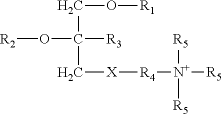
or an enantiomer or diastereomer or a pharmaceutically acceptable salt thereof, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is independently selected from the group consisting of hydrogen and methyl, to the patient, thereby treating clinically isolated syndrome, relapsing remitting multiple sclerosis (RR-MS), and/or secondary progressive multiple sclerosis (SP-MS) in the patient.

2. A method of treating a multiple sclerosis patient not adequately responding to interferon therapy comprising administering an effective amount of a tri-substituted glycerol compound of formula (I) 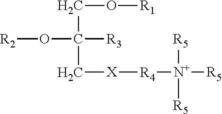
or an enantiomer or diastereomer or a pharmaceutically acceptable salt thereof, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is selected from the group consisting of hydrogen and methyl, to the patient, thereby treating multiple sclerosis in the patient.

3. The method of claim 1, wherein X is phosphate, R₁ is —(CH₂)₁₇—CH₃, R₂ is CH₃, R₃ is H, R₄ is —(CH₂)₂—, and R₅ is CH₃.

4. The method of claim 1, wherein the tri-substituted glycerol compound is administered in an amount of 10-40 mg/day.

5. The method of claim 1, wherein the tri-substituted glycerol compound is administered in combination with at least one further pharmaceutically active compound.

6. The method of claim 5, wherein the at least one further pharmaceutically active compound is selected from the group consisting of glucocorticoids, interferon-beta compounds, immunosuppressive agents, immunomodulatory compounds, monoclonal antibodies, immunoglobulin, and fumarate.

7. The method of claim 6, wherein the at least one further pharmaceutically active compound is selected from the group consisting of teriflunomid, laquinimod, and fumarate.

8. The method of claim 1, wherein the tri-substituted glycerol compound is administered orally.

9-15. (canceled)

16. The method of claim 3, wherein the tri-substituted glycerol compound is administered in combination with at least one further pharmaceutically active compound.

17. The method of claim 16, wherein the at least one further pharmaceutically active compound is selected from the group consisting of glucocorticoids, interferon-beta compounds, immunosuppressive agents, immunomodulatory compounds, monoclonal antibodies, immunoglobulin, and fumarate.

18. The method of claim 17, wherein the at least one further pharmaceutically active compound is selected from the group consisting of teriflunomid, laquinimod, and fumarate.

19. The method of claim 2, wherein X is phosphate, R₁ is —(CH₂)₁₇—CH₃, R₂ is CH₃, R₃ is H, R₄ is —(CH₂)₂—, and R₅ is CH₃.

20. The method of claim 2, wherein the tri-substituted glycerol compound is administered in an amount of 10-40 mg/day.

21. The method of claim 2, wherein the tri-substituted glycerol compound is administered in combination with at least one further pharmaceutically active compound.

22. The method of claim 21, wherein the at least one further pharmaceutically active compound is selected from the group consisting of glucocorticoids, interferon-beta compounds, immunosuppressive agents, immunomodulatory compounds, monoclonal antibodies, immunoglobulin, and fumarate.

23. The method of claim 22, wherein the at least one further pharmaceutically active compound is selected from the group consisting of teriflunomid, laquinimod, and fumarate.

24. The method of claim 2, wherein the tri-substituted glycerol compound is administered orally.

25. The method of claim 19, wherein the tri-substituted glycerol compound is administered in combination with at least one further pharmaceutically active compound.

26. The method of claim 25, wherein the at least one further pharmaceutically active compound is selected from the group consisting of glucocorticoids, interferon-beta compounds, immunosuppressive agents, immunomodulatory compounds, monoclonal antibodies, immunoglobulin, and fumarate.

27. The method of claim 26, wherein the at least one further pharmaceutically active compound is selected from the group consisting of teriflunomid, laquinimod, and fumarate.
